(19) United States
(12) Patent Application Publication
(10) Pub. No.: US 2019/0343493 A1
(43) Pub. Date: Nov. 14, 2019

(54) SAMPLE TRAY AND CONTAINER AND METHODS OF OPERATION THEREOF

(71) Applicant: GA Health & Beauty Co Limited, Shatin N.T. (HK)

(72) Inventor: Kenneth Thomas McCabe, Shatin N.T. (HK)

(21) Appl. No.: 15/978,142

(22) Filed: May 13, 2018

(51) Int. Cl. A61B 10/00; A61B 10/02

(52) U.S. Cl. CPC A61B 10/0096; A61M 1/0056; A61B 10/0283

(57) ABSTRACT

A sample collection system including a container including container walls that enclose a container cavity; a fluid inlet extending through one of the container walls; a fluid outlet extending from outside the container into the container and into the container cavity through the one of the container walls; a sample tray configured to be removably insertable through one of the container walls into the container below the fluid inlet and including a filter portion, the tray being insertable below the fluid inlet to provide a fluid path that flows from the fluid inlet through the filter portion of the sample tray to the fluid outlet during suctioning, wherein a bottom portion of the fluid outlet extends away from the one of the container walls into the container cavity below the filter portion.

### FIELD

The present system relates to a sample tray and a corresponding container for use during a suctioning procedure including an endoscopic procedure and also relates to a sample collection system including at least one sample tray and a container and methods of operation thereof.

### BACKGROUND

Suctioning procedures such as during an endoscopic procedure are performed on patients for a variety of reasons. For example, an endoscope may be utilized during a gastrointestinal procedure to detect abnormalities or growths, such as colorectal polyps. Polyps are problematic because they may be a precursor to cancer and are typically removed for a pathological review when found. The removal of polyp during a colonoscopy (a polypectomy) may be performed through use of a scalpel instrument or a specialized snare, each of which is inserted through the endoscope and is utilized to separate the polyp from other tissue.

Once the polyp is separated from the other tissue, a retrieval net or filtering device such as a polyp trap may be utilized to collect the tissue sample. The polyp trap is utilized during a suctioning procedure, to separate the solid material (e.g., a sample such as a polyp) from a suctioned fluid portion. For example, during a polypectomy after the polyp or a portion thereof is free from other tissue, a suction tube such as a catheter is inserted through the endoscope and extends out from a distal end of the endoscope.

To facilitate the separation of solid material (e.g., a tissue sample) from a suctioned fluid stream, a solid filter (e.g., filter tray such as a polyp trap) or filtering material is inserted into the flow of suctioned fluid to trap or capture the suctioned sample from the fluid stream. In the use of a filtering material, it is oftentimes difficult to separate the sample from the filtering material and it is difficult to prepare the filtering material for insertion into the suctioned fluid stream.

Alternatively, a sample tray and corresponding container such as may be utilized in a polyp trap is more convenient to utilize than the filtering material yet still has many difficulties associated with selecting and assembling the portions of the trap. While the polyp trap alleviates some of the problems associated with utilizing a filtering material, the construction is difficult, expensive to manufacture and may be difficult to properly assemble during use.

Accordingly, embodiments of the present device, system and/or method may overcome these and other disadvantages of conventional filtering systems.

### SUMMARY

The system(s), device(s), arrangements(s), method(s), etc. (hereinafter each of which will be referred to as system, unless the context indicates otherwise), described herein address problems in prior art systems. Embodiments of the present system may employ sample trapping methods which include a container and a corresponding sample tray intended for operational coupling with a suctioning device, such as a pump. During operation and after coupling to the pump, a suction is created to draw a fluid including the sample through inlet/outlet couplings of the container to trap the sample out of the fluid, wherein the sample is retained in the sample tray.

In accordance with embodiments of the present system, there is disclosed, in one aspect of the present system, a sample collection system including a container including container walls that enclose a container cavity; a fluid inlet extending through one of the container walls; a fluid outlet extending from outside the container into the container and into the container cavity through the one of the container walls; a sample tray configured to be removably insertable through one of the container walls into the container below the fluid inlet and including a filter portion, the tray being insertable such that the filter portion is positioned below the fluid inlet coupling to provide a fluid path that flows from the fluid inlet through the filter portion of the sample tray to the fluid outlet during suctioning, wherein a bottom portion of the fluid outlet extends away from the one of the container walls into the container cavity below the filter portion.

The system may include a keying structure between the container and the sample tray that ensures the sample tray is only insertable into the container in a single given orientation. The sample tray may include a rotatable structure that rotates from an orientation suitable for insertion of the sample tray into the container, to a locked position wherein the sample tray is retained in the container. The rotatable structure may be configured to provide a fluid-tight seal between the container and sample tray when the rotatable structure is in the locked position. The sample tray may include an indicium that indicates when the rotatable structure is in the locked position.

An edge of the sample tray may include an indicium that indicates when the rotatable structure is in the unlocked position. The sample tray may be one of a plurality of sample trays of the system, wherein at least two of the plurality of sample trays includes an indicium that identifies at least one of the two of the plurality of sample trays apart from at least one other of the two of the plurality of sample trays. The container may include at least one tray support structure that corresponds to a structure of the sample tray to support the sample tray in a vertical direction and allow movement of the sample tray in a horizontal direction with reference to an orientation during operation. The sample tray may be one of a plurality of sample trays of the system, wherein at least two of the plurality of sample trays includes an indicium that identifies at least one of the two of the plurality of sample trays that does not include a filter portion apart from at least one other of the two of the plurality of sample trays that includes a filter portion.

In accordance with embodiments of the present system, there is disclosed, in one aspect of the present system, a polyp collection device including a container including container walls that enclose a container cavity; a fluid inlet extending through one of the container walls; a fluid outlet extending from outside the container into the container and extending into the container cavity through the one of the container walls; and a sample tray configured to be removably insertable through one of the container walls into the container below the fluid inlet and including a filter portion, the tray being insertable such that the filter portion is positioned below the fluid inlet to provide a fluid path that flows from the fluid inlet through the filter portion of the sample tray to the fluid outlet during suctioning, wherein a bottom portion of the fluid outlet extends away from the one of the container walls into the container cavity below the filter portion. The device may include a keying structure between the container and the sample tray that ensures the sample tray is only insertable into the container in a single given orientation.

The sample tray may include a rotatable structure that rotates from an orientation suitable for insertion of the sample tray into the container, to a locked position wherein the sample tray is retained in the container. The rotatable structure may be configured to provide a fluid-tight seal between the container and sample tray when the rotatable structure is in the locked position. The sample tray may include an indicium that indicates when the rotatable structure is in the locked position. An edge of the sample tray may include an indicium that indicates when the rotatable structure is in the unlocked position. The sample tray may be one of a plurality of sample trays, wherein at least two of the plurality of sample trays includes an indicium that identifies at least one of the two of the plurality of sample trays apart from at least one other of the two of the plurality of sample trays. The container may include at least one tray support structure that corresponds to a structure of the sample tray to support the sample tray in a vertical direction and allow movement of the sample tray in a horizontal direction with reference to an orientation during operation. The sample tray may be one of a plurality of sample trays, wherein each of at least two of the plurality of sample trays includes an indicium that identifies at least one of the two of the plurality of sample trays that does not include a filter portion apart from at least one other of the two of the plurality of sample trays that includes a filter portion.

### BRIEF DESCRIPTION OF THE DRAWINGS

The present system is explained in further detail in the following exemplary embodiments and with reference to the figures, wherein the features of various exemplary embodiments shown in different figures are combinable.

In the drawings:

FIG. 1 shows a side disassembled view of portions of a sample collection system (tissue trap) in accordance with embodiments of the present system;

FIG. 2 shows a top perspective view of a portion of a container in accordance with embodiments of the present system;

FIG. 3 shows side view of an opening of the container in accordance with embodiments of the present system;

FIG. 4 shows a top view of a container portion in accordance with embodiments of the present system;

FIG. 5 shows a top view of a portion of a sample tray in accordance with embodiments of the present system;

FIG. 6 shows a perspective view of a tray locking lever separated from a sample tray in accordance with embodiments of the present system;

FIG. 7A shows a top view of a base portion of a container in accordance with embodiments of the present system;

FIG. 7B shows a perspective view of the base portion shown in FIG. 7A in accordance with embodiments of the present system;

FIG. 8A shows a top view of an alternate base portion of a container in accordance with embodiments of the present system; and

FIG. 8B shows a perspective view of the alternate base portion shown in FIG. 8A in accordance with embodiments of the present system.

### DETAILED DESCRIPTION

The following are descriptions of illustrative embodiments that when taken in conjunction with the following drawings will demonstrate the above noted features and advantages, as well as further ones. In the following description, for purposes of explanation rather than limitation, illustrative details are set forth such as shape, mating surfaces, techniques, element attributes, etc. However, it will be apparent to those of ordinary skill in the art that other embodiments that depart from these details would still be understood to be within the scope of the appended claims. Moreover, for clarity detailed descriptions of well-known devices, tools, techniques, and methods are omitted so as not to obscure the description of the present device, system and method. It should be expressly understood that the drawings are included for illustrative purposes and do not represent the entire scope of the present system. In the accompanying drawings, like reference numbers (e.g., the ten's and/or one's place digit(s) are the same) in different drawings may designate similar elements.

As utilized herein, the term “and/or” and formatives thereof should be understood to mean that only one or more of the recited elements may need to be suitably present (e.g., only one recited element is present, two of the recited elements may be present, etc., up to all of the recited elements may be present) in a device, system and/or method in accordance with the claims recitation and in accordance with one or more embodiments of the present system. In the context of the present invention, the terms “about” and “approximately” denote an accuracy that a person skilled in the art will understand to still ensure the technical effect of the feature in question. As utilized herein, the term(s) “about” and “approximately” for example may indicate a deviation from an indication (e.g., an indicated numerical value, an indicated relational size, such as about a same size, etc.) of ±20%, or ±15%, or ±10%, or ±5%, or ±1%, or ±0.5%, or ±0.1% or ±0.05%, or another tolerance that is suitable based on cost and use considerations. Furthermore, the terms “first”, “second”, “third”, etc., and/or “(a)”, “(b)”, “(c)”, “(d)”, etc., and/or “(i)”, “(ii)”, “(iii)”, “(iv)”, etc., and the like in the description and in the claims, may be, however need not necessarily be used for distinguishing between similar elements and may though is not necessarily used for describing a sequential or chronological order. It is to be understood that the terms so used are interchangeable under appropriate circumstances and that the embodiments of the invention described herein are capable of operation in other sequences than described or illustrated herein. The term “substantially” as may be utilized herein to modify an application term (e.g., “substantially homogeneous”) is intended to mean mostly and/or within reasonable engineering tolerances (e.g., mostly homogeneous and/or within reasonable engineering tolerances). For example, as utilized herein, substantially at a known position (e.g., substantially at the isocenter axis) means within reasonable engineering tolerances (e.g., ±20%, or ±15%, or ±10%, or ±5%, or ±1%, or ±0.5%, or ±0.1% or ±0.05%, etc.) of that known position. The term(s) “coupled”, “operatively coupled” and formatives thereof as utilized herein refer to a connection between devices or portions thereof that enables operation in accordance with the present system.

For the sake of clarity, embodiments of the present system will be shown and described with respect to illustrative sample tray(s)/container(s) such as may be employed during a suctioning procedure. However, it is also envisioned that embodiments of the present system may be sized and shaped differently from the illustrative examples shown. Further, it should be assumed that the container of the present system when typically used, is coupled to and/or form an input and output conduit such as two or more tubing portions such as catheters, and the like which in turn form a portion of a suctioning apparatus or are coupled thereto. Further, the dimensions and proportions of objects shown in the figures are for illustrative purposes. The drawings are not drawn to scale but are shown to illustrate portions of the present sample collection system. In accordance with the present invention, the illustrative example(s) shown are discussed herein for illustrative purposed, however should be understood to apply to other suitable systems, as applicable, without further elaboration unless noted including taking any one or more features described or shown in one or more illustrative embodiments and utilizing that same feature(s) in another embodiment shown, described or applicable without further elaboration.

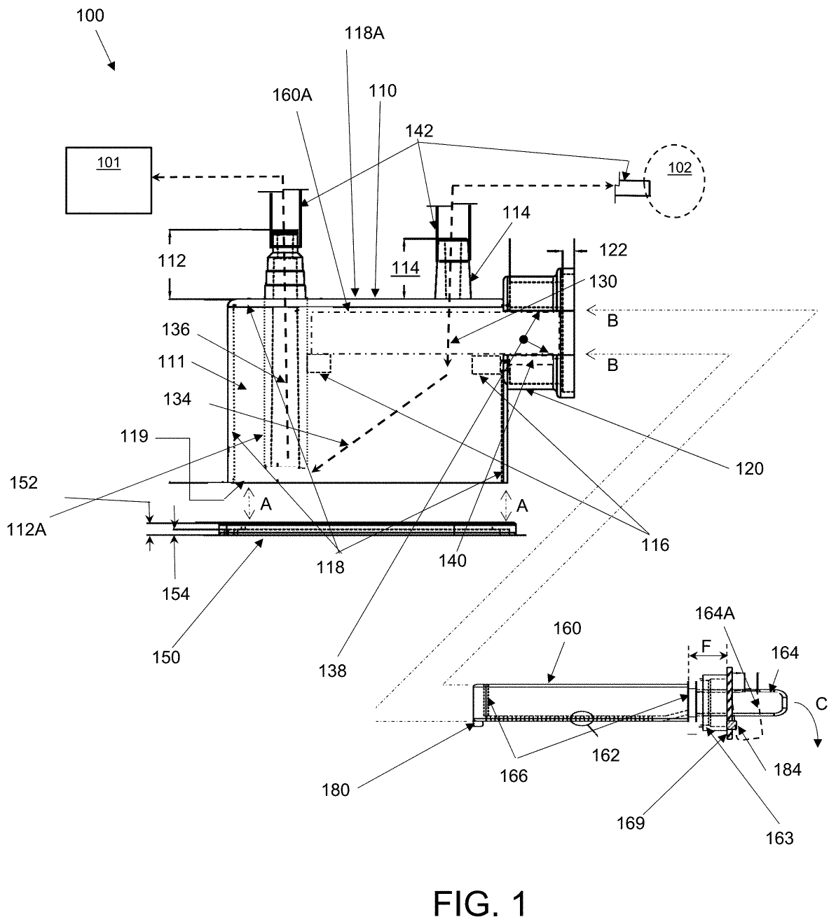

FIG. 1 shows a side disassembled view of portions of a sample collection system 100, such as a tissue trap, polyp trap, etc., in accordance with embodiments of the present system. As shown, the sample collection system 100 may include one or more of a container 110, a removable sample tray 160 and a base 150. The container 110 is shown in FIG. 1 in an upright orientation which is a typical orientation of the container 110 during a suctioning procedure. To facilitate the further discussion herein, the terms such as top, bottom, left, right, side(s), horizontal and vertical will be utilized herein and may refer to the orientation of the sample collection system 100 during operation such as shown in FIG. 1.

As shown, the container 110, prior to introduction of the sample tray 160 as is described in more detail below, is substantially a hollow, six-sided object that may be constructed (e.g., molded, such as injection and/or vacuum molding/forming and/or other forming process) of a transparent material such as a clear plastic or other transparent material. The container 110 includes an inlet 114 (e.g., an inlet coupling), an outlet 112 (e.g., an outlet coupling), a tray receptacle 120 including a receptacle lip portion 122, and container walls 118. In accordance with embodiments of the present system, the container walls 118 are formed (e.g., sized and shaped) to enclose the sample tray 160 within an interior container cavity 111. In accordance with embodiments of the present system, the container walls 118, such as shown in the embodiment in FIG. 1, substantially surround the sample tray 160 on five (5) sides leaving a bottom opening 119. In these embodiments, prior to use such as during construction, the bottom opening 119 may be sealed by the base 150. For example, during construction of the sample collection system 100 and particularly the container 110, the base 150 may be coupled (e.g., adhered, glued, laminated, welded, etc.) to the container 110 to provide a fluid-tight seal between the bottom opening 119 of the container 110 and the base 150. In FIG. 1, this coupling is shown by arrows and dashed lines “A” which indicate the coupling of the container 110 to the base 150. In accordance with further embodiments of the present system, the base may be formed together with the container.

The inlet and outlet 114, 112 may be conical shaped as shown and may have a narrowing outer diameter toward a distal end away from the container 110 to facilitate coupling suction tubing 142, such as a cannula, to the inlet and outlet 114, 112 (e.g., coupling the tubing to inlet and outlet of the container). As may be readily appreciated, other methods of connecting the suction device and outlet tubing to the respective inlet and outlet of the container may also be suitably utilized without deviating from the spirit of the present system. For example, the inlet and/or outlet may have a uniform outside diameter that is smaller than an inside diameter of the tubing. Further in accordance with embodiments, wherein the container has one or more of the inlet and outlet, the inlet and/or outlet may have other sizes and/or shapes than those illustratively shown. In accordance with embodiments of the present system, one or more of inlet and outlet tubing may be permanently affixed to the container in place of the inlet and/or outlet.

In any event, during operation the container is coupled to a suction device 101 (e.g., a pump) through the outlet of the container and the inlet of the container is coupled to tubing that extends to a desired suction site 102 (e.g., at the distal end of the endoscope). In this way, fluids such as bodily fluids and samples such as tissue samples may be drawn by the suction device 101 from the suction site 102 through the inlet and outlet of the container (e.g., the inlet and outlet 114, 112) and a filter portion 162 of the sample tray 160, positioned in effect midstream or therebetween the inlet and outlet of the container.

During operation, the suction device 101 creates a suction flow as illustrated in FIG. 1 with an inlet flow 130, container flow 134 and an outlet flow 136. The suction flow created by the suction device 101 coupled to the outlet 112, 212, 412 of the container (e.g., the outlet coupling) for example by way of the tubing 142, draws fluids and sample material (e.g., sample tissue such as a polyp or portion thereof and/or another desired tissue sample) from the suction site 102 through the container so that the sample material may be captured in the sample tray 160. In accordance with embodiments of the present system, the outlet 112, 212, 412 may include an extension portion 112A, 212A that extends into the container cavity 111 from a top portion of the container cavity (e.g., a top container wall 118A) down towards, without reaching, a bottom portion of the container cavity and/or the base. The utilization of the extension portion 112A, 212A in accordance with embodiments of the present system creates downward flow in the container cavity through the sample tray from the inlet of the container and thereby greatly simplifies the construction of the container and reduces costs associated therewith.

In accordance with embodiments of the present system, the inlet and/or outlet of the container may not be provided and in place, the tubing may be non-removably coupled to the container as discussed above. For example, in accordance with embodiments of the present system, during construction in place of the inlet and/or outlet, tubing may be coupled directly to the top container wall 118A in place of utilizing one or more of the inlet and outlet. In these embodiments, to seal the tubing to the container, an external surface of the tubing may be bonded, glued, welded, etc., to the container wall. As readily appreciated, other systems of coupling the tubing to the container (i.e., fluidically sealing the tubing to the container) may be suitably applied.

In accordance with embodiments of the present system wherein tubing is non-removably coupled directly to the container for example as an outlet (e.g., in place of a separate outlet of the container), the tubing may, though need not be inserted through the top container wall 118A into the interior of the container and may be positioned within the container cavity (e.g., container cavity 111) for example in place of the extension portion 112A, 212A. In accordance with these embodiments, wherein the tubing does extend into the container, it may be desirable that at least the tubing within the container has sufficient rigidity at least to maintain the tubing below the sample tray during a suctioning procedure, for example as shown in the figures. For example, in accordance with embodiments of the present system wherein the tubing extends into the container and operates as an outlet, a rigidity of the tubing may change in that at least a portion of the tubing within the container (e.g., a first portion) is more rigid than at least a portion of the tubing that extends outside the container (e.g., a second portion). In this way, at least the first portion of the tubing within the container that is comparatively rigid (i.e., more rigid than at least the second portion of the tubing), resists movement within the container when suction is applied. As utilized herein, the expression “resists movement” means that at least during suctioning, the first portion of the tubing resists being drawn to the sample tray (e.g., the first portion of the tubing may move during suctioning but is not drawn to touch the sample tray and/or a tubing outlet within the container cavity remains positioned below the sample tray). Further, the second portion of the tubing that is comparatively less rigid than the first portion, enables flexible placement and positioning of the second portion of the tubing to facilitate coupling of the tubing (e.g., outlet tubing) to a suction device (e.g., a fitting of the suction device wherein a vacuum is provided).

Further in accordance with embodiments of the present system, the tubing within the container may be maintained away from contact with the sample tray as desired. For example, movement of the tubing within the container may be constrained due to rigidity of the tubing as discussed above and/or by affixing the tubing to an interior portion of the container and/or by providing tabs, such as one of more of the tray support structures, that interfere with free movement of the tubing within the container, etc. In this way, it can be ensured that the fluid is properly drawn through the sample tray from below the sample tray. In accordance with one or more of these embodiments, the tubing may extend directly to the suction device (e.g., outlet tubing) and/or suctioning site (e.g., inlet tubing) or may continue to a coupling for further tubing, etc., to complete the sample collection system.

In accordance with embodiments of the present system wherein outlet tubing does extend into the container cavity and the tubing within the cavity is obstructed from movement towards the sample tray (e.g., by one or more of ledges and/or other physical features that obstruct movement of the tubing towards the sample tray within the cavity), the rigidity of the tubing within the container cavity need not change from the rigidity of the tubing outside the container cavity (e.g., the first portion of the tubing need not be relatively more rigid than the second portion of the tubing). For example, since the proper positioning of the tubing within the container need not depend on an increase in the rigidity of the tubing in embodiments wherein the tubing within the container cavity is obstructed from movement as described, the rigidity of the tubing need not, though may change from inside and outside of the container. For example, in accordance with embodiments wherein movement of the tubing is obstructed within the container cavity, the tubing rigidity may be uniform throughout the length of the tubing or a portion of the tubing that is within the container cavity may be more or less rigid than a portion of the tubing that extends away from the container.

To facilitate the sample collection system 100 and particularly the container 110, to stand upright during operation (i.e., with the inlet and outlet 114, 112 extending upward from the top container wall 118A and coupled to inlet and outlet tubing), the base 150 may have an outside perimeter that is circumferentially larger than the outside perimeter of the bottom opening 119 as shown in FIG. 1. In this way, the base 150 may be set on a flat surface during operation to assist in keeping the container 110 upright.

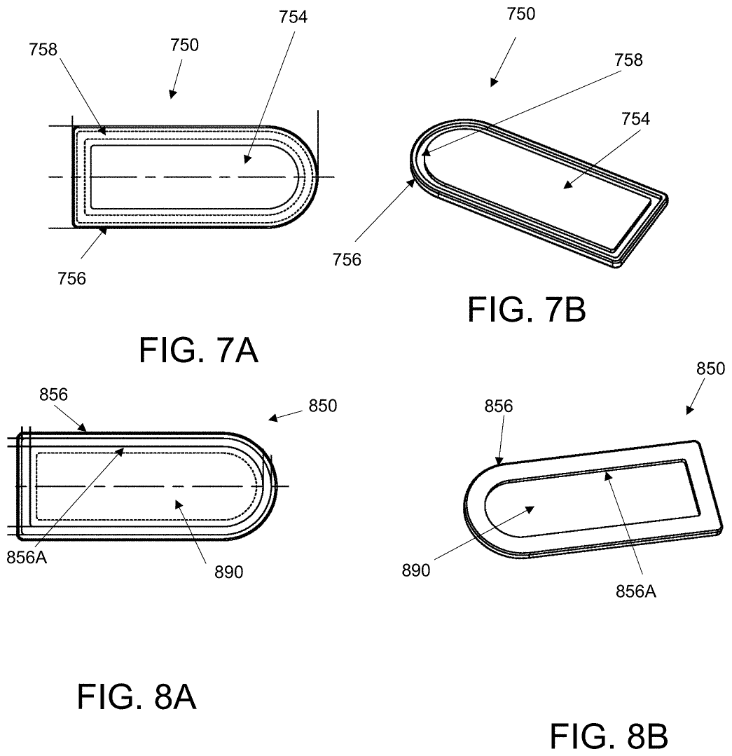

A discussion of the details of the construction of the base 150 is provided with reference to FIGS. 1, 7A, 7B, 8A, 8B in accordance with embodiments of the present system. Particularly, FIG. 7A shows a top view of a base 750 of a container in accordance with embodiments of the present system which is similarly formed as the base 150 shown in FIG. 1. FIG. 7B shows a perspective view of the base 750 shown in FIG. 7A in accordance with embodiments of the present system. FIG. 8A shows a top view of an alternate base 850 of a container in accordance with embodiments of the present system. FIG. 8B shows a perspective view of the alternate base 850 shown in FIG. 8A in accordance with embodiments of the present system

As shown in FIGS. 1, 7A, 7B, 8A, 8B, the base 150, 750, 850 has a base height 152 (i.e., extension in the vertical direction) which is provided by an upward extension of a base sidewall 756, 856. In accordance with embodiments such as shown in FIGS. 7A, 7B, the base sidewall 756 extends upward as described above and further extends around a slot 758, which in turn extends around a base extension 154, 754. Further, the base extension 154, 754 may also extend upward, thereby forming the slot 758 therebetween. Further, a thickness of the slot extending around the base 150, 750 is defined by an inner wall of the base sidewall 756 (e.g., with reference to an interior portion of the base 150, 750 that is surrounded by the base sidewall 756) and an outer wall of the base extension 154, 754. It should be readily appreciated that although in FIG. 1 the base extension 154 is shown to not extend upward as far as the base height 152, in accordance with embodiments of the present system the base extension may extend the same or higher than the base height, for example to facilitate operative coupling of the base 150 to the container 110.

In accordance with embodiments of the present system, to facilitate the coupling of the container 110 to the base 150, 750, the size and shape defining the thickness of the slot 758 may be sized and shaped to be about a same size (e.g., slightly larger) and of similar sizing and shaping as the container walls 118. In this way, the container walls 118, during coupling of the container 110 to the base 150 (see, FIG. 1), may snugly fit within the slot 758 surrounded by a portion of the base extension 754 along an interior portion of the container walls 118 and surrounded by a portion of the base sidewall 756 along an exterior portion of the container walls 118.

In the embodiments shown in FIGS. 8A, 8B, the base sidewall 856 extends upward as described above and further extends around a recess 890. In addition, a thickness of the recess 890 extending around the base 850 is defined by an inner wall 856A of the base sidewall 856. To facilitate the coupling of the container 110 to the base 850, the size and shape of the inner wall of the base sidewall 856 may be sized and shaped to be about a same size (e.g., slightly larger) and of similar sizing and shaping as an exterior surface of the container walls 118. In this way, the container walls 118, during coupling of the container 110 to the base 850 (e.g., see, FIG. 1), may snugly fit within the recess 890 surrounded by a portion of the inner wall 856A of the base sidewall 856 along an exterior portion of the container walls 118.

In accordance with further embodiments of the present system, the recess of FIGS. 8A, 8B may be substituted for a platform that extends upward higher than the upward extension of the base sidewalls 856. In these embodiments, to facilitate the coupling of the container 110 to the base 850, the size and shape of an outer wall of the platform may be sized and shaped to be about a same size (e.g., slightly smaller) and of similar sizing and shaping as an interior surface of the container walls 118. In this way, the container walls 118, during coupling of the container 110 to the base, may snugly fit around the platform of the base surrounding a portion of the base sidewall along an interior portion of the container walls 118.

In the context of the present invention, the term “snugly fit” denotes an accuracy in size and or shape between at least two surfaces that a person skilled in the art will understand will still ensure the technical effect, either alone or together with an additional suitable adhesion method, of creating a substantially fluid tight seal (e.g., a coupling) between the container and the base to ensure that the sample collection system is operational as described further herein.

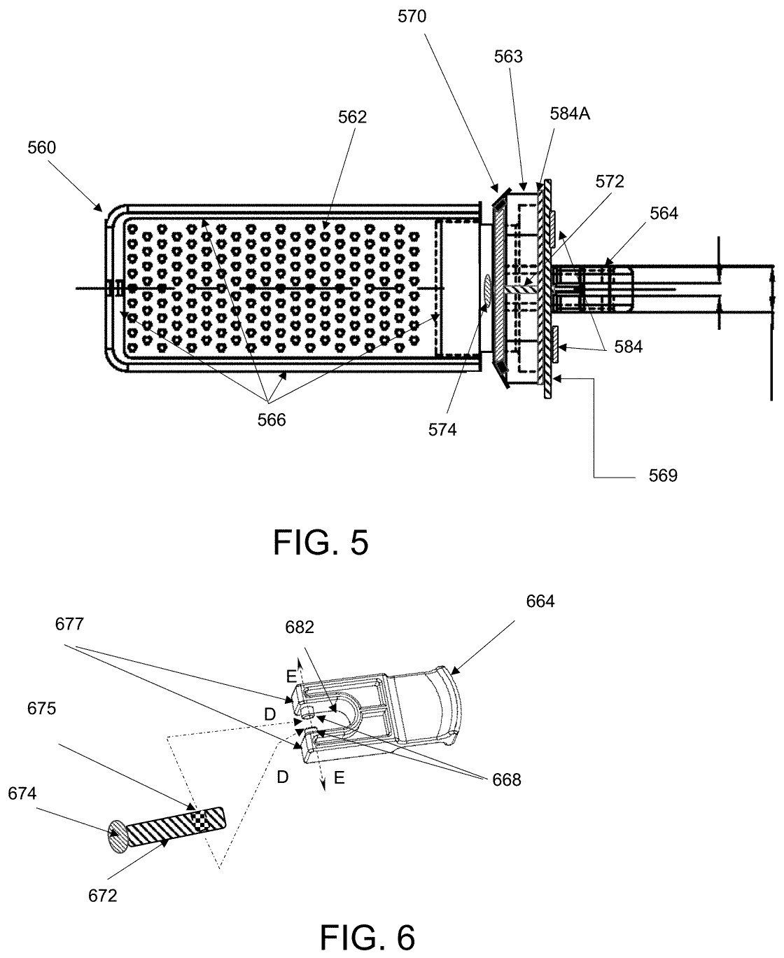

Returning to the sample tray 160, 560, in detail FIG. 5 shows a top view of a portion of a sample tray 560 in accordance with embodiments of the present system. FIGS. 1, 5 show that the sample tray 160, 560 has tray sidewalls 166, 566 that extend downward to the filter portion 162, 562. The tray sidewalls 166, 566 surround the filter portion 162, 562 on four sides while the filter portion 162, 562 forms a bottom of the sample tray. In FIG. 1, the arrows and dashed lines B illustrate an insertion direction of the sample tray 160 into the container 110. As shown by a sample tray outline 160A, the sample tray 160 is inserted into the container such that the tray sidewalls 166, 566 surround the inlet coupling 114 to ensure that the fluid flow inside the container during operation, flows from the inlet of the container through the sample tray to the outlet of the container.

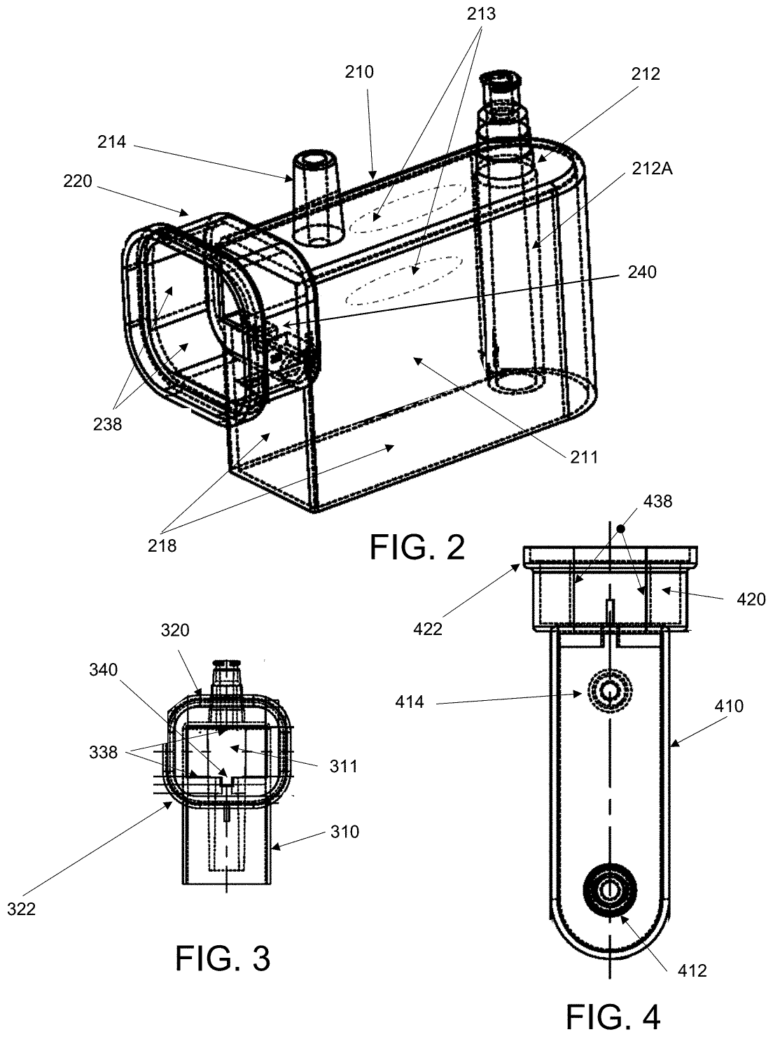

As is clear from an upper horizontal portion of the arrows and dashed lines B that are closest to the container 110 and that are shown in FIG. 1, the insertion direction of the sample tray 160, 560 is substantially horizontal throughout the insertion into the container 110, 210, 310, 410 as is shown additionally in FIGS. 2, 3, 4. For example, FIG. 2 shows a top perspective view of a portion of the container 210 in accordance with embodiments of the present system. FIG. 3 shows side view of an opening of the container 310 in accordance with embodiments of the present system. FIG. 4 shows a top view portion of the container 410 in accordance with embodiments of the present system.

To facilitate a reduction in vertical motion of the sample tray during insertion into the container, the container may include one or more tray support structures 116 (e.g., one or more ledges, lips, slides, etc.) that support the sample tray during insertion and thereafter, for example, during operation of the sample collection system. As readily appreciated, the one or more tray support structures 116 are sized and shaped to vertically support the sample tray without interfering with a horizontal insertion and operation of the sample tray, and particularly operation of the filter portion 162, 562. These features of the container may cooperate with corresponding features of the sample tray 160, 560, such as the tray sidewalls 166, 566 to facilitate support in the vertical direction. In addition, the features of the container may cooperate with corresponding features of the sample tray to ensure that the sample tray is only insertable into the container in a single given desired orientation (e.g. the orientation shown in FIG. 1). For example, the features of the container may interfere with features of the sample tray when the sample tray is not inserted into the container in the single given desired orientation.

Further, the tray receptacle 120 may have internal wall feature(s) 138, 238, 338, 438 that are sized and shaped to cooperate with a sample tray stopper 163, 563 of the sample tray 160 to add vertical support of the sample tray and/or to ensure that the sample tray is only insertable into the container in the single given desired orientation. In addition, the tray receptacle 120 may terminate at a distal end at the receptacle lip portion 122, 322, 422 and may be sized and shaped to cooperate with a tray end wall 169, 569 of the sample tray 160 to add vertical and horizontal support of the sample tray and/or to ensure that the sample tray is only insertable into the container in the single given desired orientation. During insertion of the sample tray into the container, the tray end wall 169, 569 may interact with the receptacle lip portion 122, 322, 422 and wall features of the receptacle lip portion 122, 322, 422 to stop the horizontal insertion when the sample tray is fully inserted into a suitable position for operation and/or is inserted into the single given desired orientation.

In accordance with embodiments of the present system, a portion of the tray receptacle 120, 220, 320, 420 may include one or more keyway(s) 140, 240, 340 that interact with correspondingly sized and shaped keying 180 of the sample tray. In this way, the keying between the container and the sample tray may be utilized to ensure that only a compatible sample tray is inserted into the container. For example, a sample tray that is improperly sized for a given container, may have keying that is incompatible with the given container (e.g., the keying may interfere with keying features of the improper given container). In embodiments wherein two or more sample trays and one or more corresponding containers together form a given sample collection system, keying between the two or more sample trays and the one or more corresponding containers may ensure that only compatible sample trays (e.g., sample trays within the given sample collection system) are insertable into containers that correspond to the sample trays. For example, keying between the two or more sample trays and the compatible container may not interfere with insertion of the one or more compatible sample trays into the compatible container. In contrast, keying between one or more sample trays that are not intended for the given sample collection systems one of more containers may interfere with insertion of the one or more sample trays that are not intended for the given sample collection system.

In addition, in accordance with embodiments of the present system keying between a sample tray and a compatible container may be cooperatively positioned (e.g., positioned on both the sample tray and the compatible container), cooperatively sized (e.g., sized on both the sample tray and the compatible container) and/or cooperatively structured (e.g., structured on both the sample tray and the compatible container) to ensure that the sample tray is only insertable into the container in a single given (desired) orientation.

It should be readily appreciated that although the keying 180 is shown extending from sample tray 160, in a similar fashion the keying 180 may form an indentation in the sample tray 160 as long as the keyway has a corresponding structure (e.g., a corresponding elevation). In accordance with embodiments of the present system, the keying for example on the container may extend from any side and/or may be provided any given shape on the container that corresponds to keying that is correspondingly positioned and/or shaped on the sample tray. The keying may also be provided in accordance with embodiments of the present system, for example on one or more of the tray receptacle, the receptacle lip portion, the sample tray stopper, the tray walls, and/or the sample tray stopper together with other corresponding structures.

In operation, the keying between the container and the sample tray may facilitate proper insertion and/or proper orientation of the sample tray with reference to the orientation of the container. For example, in accordance with embodiments of the present system the keying may be utilized to ensure that the sample tray is only insertable into the container in a single desired orientation (e.g., with the filter portion extended away from the container walls). In addition or in place of the keying, a size and/or shape of the sample tray may be formed to only be insertable into the container in a single orientation, for example through forming of a tapered wall structure of the sample tray that corresponds to a tapered wall structure of the tray receptacle and/or other corresponding structures such as discussed above.

As for example shown in FIGS. 2, 3, the tray receptacle 220, 320 forms an opening to a container cavity 211, 311 into which the sample tray is inserted. During insertion of the sample tray into the container, a structure 164, 564, 664 such as a locking lever (e.g., see, FIGS. 1, 5, 6), may be grasped by a user to support the sample tray during the insertion. Following insertion of the sample tray, it is desirable that together with the corresponding container wall which includes the opening, and/or receptacle lip portion, a fluid tight seal is formed between the corresponding container wall and the sample tray. In accordance with embodiments of the present system, the sample tray stopper 163, 563 may interact with the tray receptacle and/or the receptacle lip portion to facilitate the fluid tight seal on insertion of the sample tray into the container.

In accordance with embodiments of the present system, the structure 164 may operate as a locking lever that is rotatable (e.g., see arrow C indicated in FIG. 1) between a horizontal position as shown in FIG. 1 and a vertical position shown as shown by dashed lines 164A of the structure 164. In the embodiment shown in FIG. 1, the vertical position shown is termed a “locked position”. As utilized herein, the term locked position is intended to connote a position wherein a fluid tight seal between the container and the sample tray is originated or enhanced through operation and rotation of the structure/locking lever 164, 564.

Further operation of the locking lever is provided with reference to FIG. 6 which shows a perspective view of a tray locking lever 664 separated from the sample tray in accordance with embodiments of the present system. As shown, the locking lever 664 has a U-shaped opening 682 at a portion of the locking lever 664 that is positioned adjacent to the tray end wall 569 when attached to the sample tray. To facilitate attachment of the locking lever 664 to the sample tray, such as the sample tray 560, the sample tray 560 may include a tab 572, 672 that includes a feature 675, such as indents 572, 672 that extend into the tab, or a hole that extends therethrough. As made clear by the arrows and dashed lines D shown in FIG. 6, when assembled the tab 572, 672 is retained by the locking lever 564, 664 through interaction (e.g., insertion) of circular tabs 668 of the locking lever with the feature 675 of the tab. When assembled, the interaction of the circular tabs 668 of the locking lever with the feature 675 of the tab 572 672, may provide a pivoting motion between the locking lever and the tab about a pivot axis E. In accordance with embodiments of the present system, the tabs of the locking lever may be replaced, for example with a pin that passes through the locking lever and the tab, thereby enabling the pivoting of the locking lever as described. Other systems may also be suitably utilized for coupling the locking structure to the tab in accordance with embodiments of the present system.

In operation, the tab 572, 672 may be slidably received by the sample tray stopper and may terminate at a distal end with a tab extension 574, 674. As is shown in the figures, the tab extension 574, 674 in this embodiment has a larger outside dimension than the tab 572, 672 such that the tab extension 574, 674 cannot be slidably received into the sample tray stopper 563. In accordance with further embodiments of the present system, the tab extension may not be utilized and the tab 572, 672 for example may be directly attached to one of the tray sidewalls 566 to retain the tab 572, 672 to the sample tray 560. As readily appreciated, other systems of retaining the tab to the sample tray may be suitably employed.

In any event, when the sample tray is inserted into the container and particularly when the sample tray including the sample tray stopper is fully inserted into the tray receptacle (e.g., inserted as far as it will go into the tray receptacle), a fluid tight seal may be created between the container and the sample tray. For example, one or more of the sample tray stopper and a sealing surface such as a compression seal may form a fluid tight seal with an interior wall of the tray receptacle on insertion of the sample tray into the container.

However in accordance with embodiments of the present system, to enhance or create the fluid tight seal, the locking lever may be rotated from an unlocked position (e.g., the horizontal position for example as shown in FIG. 1 for the structure 164) to a locked position (e.g., the vertical position as shown in FIG. 1 as the structure 164A). As may be readily appreciated, in accordance with embodiments of the present system, the pivot of the locking lever may be vertically (e.g., from a middle or left position to a right locked position or from a middle or right position to a left locked position).

Through interaction of the tray end wall 569 and locking lever wall portions 677, the tab 572, 672 (and/or the tray sidewalls in these further embodiments) may be drawn towards the locking lever when the locking lever is pivoted from an unlocked position to a locked position. In accordance with embodiments of the present system, the distance that the tab moves towards the locking lever may be adjusted by adjusting one or more of the location of the pivot axis and the extension of the locking lever wall portions 677.

For example, a distance between the tray end wall 169 and a tray sidewall 166 that is adjacent to the tray end wall 169 is shown with the locking lever in an unlocked position as a distance F indicated with arrows in FIG. 1. In accordance with embodiments of the present system, when the locking lever is rotated from the unlocked position to a locked position (e.g., see the arrow C indicated in FIG. 1), the distance F may be reduced from that shown in FIG. 1 by the tab being drawn towards the locking lever as the locking lever is rotated to the locked position (e.g., see the dashed lines 164A). In accordance with further embodiments of the present system, the distance F may increase when the locking lever is rotated to the locked position and thereby a fluid tight seal may be formed.

In accordance with embodiments of the present system and regardless of whether the tab is drawn towards or moved away from the locking lever when the locking lever is moved to a locking position, through interaction of the tray end wall 569 and the locking lever wall portions 677, the tab extension 574, 674 (and/or the tray sidewall in these further embodiments) may interact with a sealing device such as a compression seal 570 (e.g., may press against an interior surface of the compression seal), or other suitable sealing mechanism, thereby providing a fluid tight seal.

For example, in an embodiment wherein the tab is drawn towards the locking lever when the locking lever is rotated towards the locked position, the sample tray stopper 563 may interact with the compression seal 570, thereby expanding the compression seal 570 as is known (e.g., through interaction with the sample tray stopper 563 moving against the compression seal when a structure, such as the structure/locking lever 164, is moved from an unlocked to a locked position). In accordance with these embodiments, the sample tray stopper 563 interacting with the compression seal 570 increases an outside diameter of the compression seal when the tab is rotated to the locked position, thereby sealing (e.g., a fluid tight seal) the sample tray to the container and/or enhancing the seal therebetween. As readily appreciated, a sealing device/system such as the illustrative compression seal shown, an O-ring, a gasket and/or other sealing device may be comprised of many different suitable materials including one or more of silicone, latex, rubber, foam, foam, plastic (soft or otherwise) ploy-vinyl chloride (PVC), thermos-plastic elastomer (TPE), etc.

Other systems for providing a seal may be utilized in accordance with embodiments of the present system and may be used independent of or together with other sealing systems (e.g., a compression seal). For example, an O-ring may be positioned on the sample tray stopper to form a seal for example together with the internal wall features (e.g., see, FIG. 1, the internal wall features 138). In accordance with these or further embodiments, an O-ring may also or alternatively be positioned on the tray receptacle such as the internal wall features (e.g., see, internal wall features 138) and may form and/or assist in forming a fluid tight seal with the sample tray stopper upon insertion of the sample tray stopper into the tray receptacle. Further, a gasket may be positioned on the wall of the receptacle lip portion, such as within a recess on the wall of the receptacle lip portion, to form a seal for example together with the one or more of the sample tray stopper and/or the tray end wall.

In this way, when a vacuum is introduced into the container via suction being applied to the outlet of the container, the only substantial entry for fluids will be via the inlet of the container and not through the fluid tight seal. In accordance with embodiments of the present system, to enhance the fluid flow through the container, a top portion of the sample tray may touch, seal or partially seal to an interior surface of the top container wall 118A to ensure that the fluid flow during operation is through the filter portion and not around the sample tray.

In accordance with embodiments of the present system, the filter portion 162, 562 includes perforations that extend through the filter tray thereby creating a filtering surface for retention of tissue samples as desired. The perforations may be formed in a pattern and may have a bore diameter that is selected to facilitate retention of samples of a desired size. For example, should a sample of a minimum size be desired, the bore diameter may be selected to retain samples of the minimum desired size. As may be readily appreciated, different sample trays may have different bore diameters to facilitate retention of a desired minimum size sample. Further, the filter portion may extend substantially horizontally as shown in the figures or may also have portions which ascend or descend, upward or downward at a slope to facilitate retention of the sample at a desired portion of the filter portion, for example spaced away from the tray sidewalls, and/or to help in separating samples that are retained by the sample tray. In accordance with embodiments of the present system, at least a portion of the tray sidewalls may slope down to the filter portion to provide rounded corners to the tray which may facilitate removal of a sample from the sample tray. Further, the filter portion and/or one or more of the tray sidewalls may include a marked scale to facilitate a determination of a given retained sample sizing (e.g., a marked millimeter scale).

In accordance with embodiments of the present system, to facilitate identification of a suitable filter tray for a desired sample size, one or more sample trays may include a tray identifier 184, 584 that may serve within the sample collection system to identify differently formed sample trays. While the tray identifier 184, 584 is illustratively shown positioned on the tray end wall, in accordance with embodiments of the present system, the tray identifier 184, 584 may be positioned on one or more different parts of the sample tray and/or more than one tray identifier may be present on the sample tray without departing from embodiments of the present system.

In accordance with embodiments of the present system, the tray identifier 184, 584 may utilize an indicium (e.g., an alphanumeric indication, a pictographic indication, a color-coded indication, an embossing and/or stamping (e.g., into the sample tray stopper), and/or different shaped structures) that indicates at least one of a suitability to the container (e.g., proper sizing for the sample tray to the container), a filter portion configuration, bore sizing, a suitability for a given desired sample-type, a fluid type, and/or a given portion of the suctioning procedure. For example, in accordance with embodiments of the present system, different indications on different sample trays may be utilized to indicate different parts of the suctioning procedure to facilitate identification of, for example, different portions of an intestine wherein the samples, such as polyps, were recovered during a gastrointestinal procedure.

Further, the tray identifier (e.g., an orange color) may be utilized to indicate a sample tray that does not include a filter portion (e.g., a bypass tray) that may be utilized to facilitate suctioning of fluids at a time when it is not desired to retain a sample. In this way, the tray identifier may indicate that the container is not in a trapping mode (e.g., a mode wherein it is not desired to retain a sample).

In accordance with embodiments of the present system, the tray identifier may also or alternatively be positioned on the locking lever, the sample tray stopper, the tray sidewalls and/or the container, for example to indicate a compatibility with one or more sample trays. In accordance with further embodiments of the present system, the tray identifier may be an indicium such as a color (e.g., a red color), for example positioned on an end of the tab close to the sample tray stopper (e.g., see, FIG. 5, tray identifier 584A) and/or may be positioned along an edge of the tray end wall. In these embodiments, when the sample tray is inserted into the tray receptacle prior to the sample tray being locked, the tray identifier 584A may be visible for example directly adjacent to the receptacle lip portion. In embodiments where the tray identifier is a color, such as the color red, the showing of a red color that is visible beyond the receptacle lip portion, may be utilized as an indication that the tray receptacle has not yet been locked (e.g., the tab has not been pivoted to a locked position). In these embodiments, when the tab is pivoted from an unlocked to a locked position and the sample tray thereby is drawn further into the tray receptacle as described above, the receptacle lip portion may now cover the tray identifier, for example covering the red color. In these embodiments, when the locking lever is in a locked position, the red color is no longer visible beyond the receptacle lip portion thereby providing a visual indication that the sample tray is locked. For example, in these or further embodiments, the tray receptacle and/or the receptacle lip portion may be opaque and/or may be a different color (e.g., a green color) than the tray identifier to cover the red color of the tray identifier 584A when the sample tray is locked.

In accordance with further embodiments of the present system, the tray identifier 584A may be a transparent color, such as a transparent yellow which may be visible beyond the edge of the receptacle lip portion when the sample tray is unlocked. Further, the receptacle lip portion may also include a transparent color, such as a transparent blue color that is also visible when the sample tray is unlocked. However, in these embodiments when the sample tray is locked, the transparent yellow color of the tray identifier may be visually mixed with the transparent blue color of the receptacle lip portion so that a combined transparent green color is visible when the sample tray is locked. In this way, a clear indication is provided when the sample tray is unlocked (e.g., by the visual indication of the separate yellow and blue colors) and when the sample tray is locked (e.g., by the visual indication of the combined green color).

The different tray identifiers may be provided for different suction applications and/or locations of suction. For example, in a sample collection system wherein a plurality of sample trays are provided, different tray identifiers such as different colors, numbers letters, icons, etc., may be provided on each of the plurality of trays to help with identifying where the samples came from and/or to help identify when/where a bypass was utilized. For example, a different tray identifier may be utilized on each sample tray to help localize where samples were retrieved.

In operation, the tray identifier 184, 584 simplifies identification of a suitable tray from amongst a plurality of sample trays included in the sample collection system and may be utilized to ensure that only a proper and desired sample tray is inserted into the container. Further, the tray identifier 184, 584 may be utilized after insertion of the sample tray into the container to verify that the proper sample tray is inserted into the container. In accordance with further embodiments of the present system, the container may include a similar identifier as the tray identifier (e.g., a similar alphanumeric, pictographic, and/or color-coded indication) to ensure that only a suitable sample tray (e.g., a compatible sample tray) is inserted into the container.

As illustratively shown in FIG. 2, the container walls may include one or more magnifier portions 213 to facilitate an examination of a sample that is retained by the sample tray within the container. In accordance with embodiments of the present system, the one or more magnifier portions 213 may be positioned, sized and shaped as desired to enhance viewing of selected portions of the filter portion wherein it is expected that a sample will be retained during suctioning.

Further variations of the present system would readily occur to a person of ordinary skill in the art and are encompassed by the following claims. Finally, the above-discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art without departing from the broader and intended spirit and scope of the present system as set forth in the claims that follow. In addition, any section headings included herein are intended to facilitate a review but are not intended to limit the scope of the present system. Accordingly, the specification and drawings are to be regarded in an illustrative manner and are not intended to limit the scope of the appended claims.

In interpreting the appended claims, it should be understood that:

a) the word “comprising” does not exclude the presence of other elements or acts than those listed in a given claim;

b) the word “a” or “an” preceding an element does not exclude the presence of a plurality of such elements;

c) any reference signs in the claims do not limit their scope;

d) several “means” may be represented by the same item or hardware or software implemented structure or function;

e) any of the disclosed elements may be comprised of hardware portions (e.g., including discrete and integrated electronic circuitry), software portions (e.g., computer programming), and any combination thereof;

f) hardware portions may be comprised of one or both of analog and digital portions;

g) any of the disclosed devices or portions thereof may be combined together or separated into further portions unless specifically stated otherwise;

h) no specific sequence of acts or steps is intended to be required unless specifically indicated;

i) the term “plurality of” an element includes two or more of the claimed element, and does not imply any particular range of number of elements; that is, a plurality of elements may be as few as two elements, and may include an immeasurable number of elements; and

j) the term and/or and formatives thereof should be understood to mean that only one or more of the listed elements may need to be suitably present in the system in accordance with the claims recitation and in accordance with one or more embodiments of the present system.

What is claimed is:

1. A sample collection system comprising:
a container including container walls that enclose a container cavity;
a fluid inlet extending through one of the container walls;
a fluid outlet extending from outside the container into the container and extending into the container cavity through the one of the container walls;
a sample tray configured to be removably insertable through one of the container walls into the container below the fluid inlet and including a filter portion, the tray being insertable such that the filter portion is positioned below the fluid inlet to provide a fluid path that flows from the fluid inlet through the filter portion of the sample tray to the fluid outlet during suctioning, wherein a bottom portion of the fluid outlet extends away from the one of the container walls into the container cavity below the filter portion.

2. The system of claim 1, comprising a keying structure between the container and the sample tray that ensures the sample tray is only insertable into the container in a single given orientation.

3. The system of claim 1, wherein the sample tray comprises a rotatable structure that rotates from an orientation suitable for insertion of the sample tray into the container, to a locked position wherein the sample tray is retained in the container.

4. The system of claim 3, wherein the rotatable structure is configured to provide a fluid-tight seal between the container and sample tray when the rotatable structure is in the locked position.

5. The system of claim 3, wherein the sample tray includes an indicium that indicates when the rotatable structure is in the locked position.

6. The system of claim 3, wherein an edge of the sample tray includes an indicium that indicates when the rotatable structure is in the unlocked position.

7. The system of claim 1, wherein the sample tray is one of a plurality of sample trays of the system, wherein at least two of the plurality of sample trays includes an indicium that identifies at least one of the two of the plurality of sample trays apart from at least one other of the two of the plurality of sample trays.

8. The system of claim 1, wherein the container includes at least one tray support structure that corresponds to a structure of the sample tray to support the sample tray in a vertical direction and allow movement of the sample tray in a horizontal direction with reference to an orientation during operation.

9. The system of claim 1, wherein the sample tray is one of a plurality of sample trays of the system, wherein at least two of the plurality of sample trays includes indicia that identifies at least one of the two of the plurality of sample trays that does not include a filter portion apart from at least one other of the two of the plurality of sample trays that includes a filter portion.

10. A polyp collection device comprising:
a container including container walls that enclose a container cavity;
a fluid inlet extending through one of the container walls;
a fluid outlet extending from outside the container into the container and extending into the container cavity through the one of the container walls;
a sample tray configured to be removably insertable through one of the container walls into the container below the fluid inlet and including a filter portion, the tray being insertable such that the filter portion is positioned below the fluid inlet to provide a fluid path that flows from the fluid inlet through the filter portion of the sample tray to the fluid outlet during suctioning, wherein a bottom portion of the fluid outlet extends away from the one of the container walls into the container cavity below the filter portion.

11. The device of claim 10, comprising a keying structure between the container and the sample tray that ensures the sample tray is only insertable into the container in a single given orientation.

12. The device of claim 10, wherein the sample tray comprises a rotatable structure that rotates from an orientation suitable for insertion of the sample tray into the container, to a locked position wherein the sample tray is retained in the container.

13. The device of claim 12, wherein the rotatable structure is configured to provide a fluid-tight seal between the container and sample tray when the rotatable structure is in the locked position.

14. The device of claim 12, wherein the sample tray includes an indicium that indicates when the rotatable structure is in the locked position.

15. The device of claim 12, wherein an edge of the sample tray includes an indicium that indicates when the rotatable structure is in the unlocked position.

16. The device of claim 10, wherein the sample tray is one of a plurality of sample trays, wherein at least two of the plurality of sample trays includes an indicium that identifies at least one of the two of the plurality of sample trays apart from at least one other of the two of the plurality of sample trays.

17. The device of claim 10, wherein the container includes at least one tray support structure that corresponds to a structure of the sample tray to support the sample tray in a vertical direction and allow movement of the sample tray in a horizontal direction with reference to an orientation during operation.

18. The device of claim 10, wherein the sample tray is one of a plurality of sample trays, wherein each of at least two of the plurality of sample trays includes an indicium that identifies at least one of the two of the plurality of sample trays that does not include a filter portion apart from at least one other of the two of the plurality of sample trays that includes a filter portion.
